(19) United States
(12) Patent Application Publication
(10) Pub. No.: US 2022/0039646 A1
(43) Pub. Date: Feb. 10, 2022

(54) DETERMINATION OF A REFRACTIVE ERROR OF AN EYE

(71) Applicant: Carl Zeiss Vision International GmbH, Aalen (DE)

(72) Inventors: Arne Ohlendorf, Tübingen (DE); Alexander Leube, Aalen (DE); Siegfried Wahl, Donzdorf (DE)

(21) Appl. No.: 17/508,629

(22) Filed: Oct. 22, 2021

Related U.S. Application Data
(63) Continuation of application No. PCT/EP2020/061207, filed on Apr. 22, 2020.

(30) Foreign Application Priority Data
Apr. 23, 2019 (EP) 19 170 558.1

(51) Int. Cl. A61B 3/032; A61B 3/00; A61B 3/14; A61B 3/11

(52) U.S. Cl. CPC A61B 3/032; A61B 3/0058; A61B 3/112; A61B 3/14; A61B 3/0033

(57) ABSTRACT

A method, a device, and a computer program for determining a refractive error of at least one eye of a user are disclosed, as well as a method for manufacturing a spectacle lens for the user. The method entails: displaying a periodic pattern on a screen, wherein a parameter of the periodic pattern includes at least one spatial frequency, wherein the parameter of the periodic pattern is varied; detecting a reaction of the user indicating that the user is able to perceive the periodic pattern; determining a point in time at which the user perceives the periodic pattern; and determining a value for the refractive error of the eye or eyes of the user from the periodic pattern at that point in time, wherein the value for the refractive error is determined from the at least one spatial frequency, determined at the point in time, of the periodic pattern.

### CROSS REFERENCE TO RELATED APPLICATIONS

This application is a continuation application of international patent application PCT/EP2020/061207, filed Apr. 22, 2020, designating the United States and claiming priority from European patent application EP 19170558.1, filed Apr. 23, 2019, and the entire content of both applications is incorporated herein by reference.

### TECHNICAL FIELD

The present disclosure relates to a method, an apparatus, and a computer program for determining a refractive error of at least one eye of a user, and to a method for producing a spectacle lens for at least one eye of the user.

### BACKGROUND

The related art has disclosed methods for determining refractive errors of an eye of a user. Here, the term “refraction” denotes a refraction of light in the eye of the user which is experienced by a light beam incident in the interior of the eye through the pupil. For the determination of the refraction, optotypes, typically in the form of numerals, letters or symbols, are usually provided on a board or a visual display unit with a defined size for a given distance and are observed by the user. By having available a number of spectacle lenses with known properties and by guiding the user through a questionnaire process, it is possible to subjectively determine the defocusing of the eye of the user and to determine which, e.g., spherocylindrical configuration of the spectacle lens leads to a substantial compensation of the refractive error of the eye and hence to an image quality for the user that is as optimal as possible. Defocusing of the eye of the user can lead to a refraction error (ametropia) of the user, in particular to nearsightedness (myopia) or farsightedness (hyperopia).

U.S. 2012/0019779 A1 discloses a method for capturing visual functions, comprising a stimulation of an optokinetic nystagmus by presenting a visual stimulus to a user; varying a first parameter of the visual stimulus; varying a second parameter of the visual stimulus; and using the varied visual stimulus to determine a threshold stimulus for the optokinetic nystagmus, wherein the first and the second parameter are selected from a group of parameters comprising a pattern for the visual stimulus, a width of the visual stimulus, a distance between the visual stimulus and the patient, a spatial frequency of the visual stimulus, a rate of change or temporal frequency of the test surface of the visual stimulus, and a contrast between elements of the visual stimulus.

U.S. 2013/0176534 A1 discloses a method for adaptively determining a model for the visual performance of a user, wherein the user is subjected to a multiplicity of tests. Each test comprises identifying a stimulating pattern, generating the pattern on a display, determining whether the pattern generates an optokinetic nystagmus, updating the model in order to include the results from the examination of the optokinetic nystagmus, and determining whether the updated model is acceptable. The tests can be repeated iteratively until the model for the visual performance of the user is acceptable.

U.S. 2014/0268060 A1 discloses an apparatus and a method for determining the refraction of an eye and an astigmatism using a computer visual display unit. To this end, an optotype is displayed on the visual display unit and a value for the size of the optotype at which the optotype is just no longer identifiable by the user is established by varying the size of the optotype displayed on the visual display unit.

WO 2018/077690 A1 discloses apparatuses and a computer program that can be used to determine the spherocylindrical refraction of an eye. To this end, a component with an adjustable optical unit is provided, the latter being able to be adjusted in respect of its refractive power by way of a refractive power setting device. Then, the spherocylindrical refraction is determined from the setting of the refractive power setting device in different orientations of a typical direction of the optical unit or typical direction of optotypes.

### SUMMARY

It is an object of the present disclosure to provide a method, an apparatus, and a computer program for determining a refractive error of at least one eye of a user, and a method for producing a spectacle lens for at least one eye of the user, which at least partly overcome the presented disadvantages and limitations of the related art.

In particular, the present method, apparatus and computer program should facilitate ascertainment of a defocusing of the at least one eye of the user in order to determine the refractive error of the at least one eye of the user therefrom. In this case, the ascertainment of the defocusing of the at least one eye of the user should be able to take place without specialist equipment and should therefore also be able to be carried out by non-specialists.

This object is achieved by a method, a computer program and an apparatus for determining a refractive error of at least one eye of a user and by a method for producing a spectacle lens for at least one eye of the user, wherein a reaction of the user is captured with an input unit. Exemplary embodiments, which can be realized individually or in combination, are presented below.

Hereinafter the terms “exhibit,” “have,” “comprise” or “include” or any grammatical deviations therefrom are used in a non-exclusive way. Accordingly, these terms can refer either to situations in which, besides the feature introduced by these terms, no further features are present, or to situations in which one or more further features are present. For example, the expression “A exhibits B,” “A has B,” “A comprises B,” or “A includes B” can refer both to the situation in which no further element aside from B is provided in A, that is to say to a situation in which A consists exclusively of B, and to the situation in which, in addition to B, one or more further elements are provided in A, for example element C, elements C and D, or even further elements.

In a first aspect, the present disclosure relates to a method for determining a refractive error of at least one eye of a user. The method comprises the following steps a) to d), typically in the stated sequence. Another sequence is also possible in principle. In particular, the method steps could also be performed entirely or partially at the same time. It is furthermore possible for individual, multiple or all steps of the method to be performed repeatedly, in particular more than once. In addition to the stated method steps, the method may also comprise further method steps.

The method for determining the refractive error of at least one eye of a user comprises the steps of:
a) representing at least one symbol on a visual display unit, wherein at least one parameter of the at least one symbol represented on the visual display unit is varied;
b) capturing a reaction of the user depending on the at least one symbol represented on the visual display unit;
c) establishing a point in time at which a recognizability of the at least one symbol represented on the visual display unit by the user is evident from the reaction of the user; and
d) determining a value for the refractive error of the at least one eye of the user from the at least one parameter defined at the point in time,

wherein the at least one symbol represented on the visual display unit is at least one periodic pattern, wherein the at least one parameter of the pattern represented on the visual display unit comprises at least one spatial frequency, and wherein the value for the refractive error is determined from the spatial frequency of the at least one pattern defined at the point in time.

The method proposed herein for determining a refractive error of at least one eye of a user is suitable, in particular, for use in a method for producing a spectacle lens for the at least one eye of the relevant user. Pursuant to the standard DIN EN ISO 13666:2013-10, also referred to as the “standard” below, sections 8.1.1 and 8.1.2, a “spectacle lens” is understood to mean an ophthalmic lens which, within the scope of the present disclosure, should serve to correct a refractive error of the eye, with the ophthalmic lens being worn in front of the eye of the user but not in contact with the eye.

In the context of the present disclosure, the term “spectacles” denotes any element which comprises two individual spectacle lenses and a spectacle frame, the spectacle lens being provided for insertion into a spectacle frame that is selected by a user of the spectacles. Instead of the term “wearer” used here, one of the terms “subject,” “spectacle wearer,” “user” or “subject” can also be used synonymously.

In the present disclosure, refractive error is understood to mean suboptimal refraction of light in at least one eye, in the case of which the image plane of the eye of light rays coming from infinity is not located at the point of intersection of all light rays coming from infinity. In this case, as a spherocylindrical refractive error, the refractive error typically comprises a spherical deviation and a cylindrical deviation and its axis. The refractive error is determined for distance vision, typically analogous to DIN 58220-5:2013-09, section 5, table 1, for a test distance between the at least one symbol and the entry pupil of the eye of ≥4 m, with a maximum deviation of ±3%, and/or for near vision, typically for a test distance between the at least one symbol and the entry pupil of the eye of <4 m or further typically, analogous to DIN 58220-5:2013-09, section 5, table 1, for a test distance between the at least one symbol and the entry pupil of the eye of 0.400 m or of 0.333 m or of 0.250 m, in each case with a maximum deviation of ±5%. Furthermore, the refractive error can also be determined for intermediate vision, typically analogous to DIN 58220-5:2013-09, for a test distance between the at least one symbol and the entry pupil of the eye of 1.000 m or of 0.667 m or of 0.550 m, in each case with a maximum deviation of ±5%.

The method according to the disclosure for determining a refractive error of at least one eye can be used if the refractive error of the at least one eye of the user has been corrected, for example by means of at least one correction lens, i.e., pursuant to the standard, section 8.1.3, a spectacle lens with dioptric power. Should the refractive error of the at least one eye be corrected, the method according to the disclosure can be used for example to check whether a change in the refractive error is present. If a change in the refractive error is present, the method according to the disclosure can be used to determine the change in the refractive error.

The method according to the disclosure for determining a refractive error of at least one eye can furthermore be used if a possibly present refractive error of the at least one eye has not been corrected, for example by means of at least one correction lens. By means of the method according to the disclosure it is furthermore possible to establish whether a refractive error of the at least one eye is even present. In the case of a known refractive error, it is furthermore possible to ascertain the change in the refractive error without the known refractive error being corrected to this end, for example by means of a correction lens. The method according to the disclosure for determining the refractive error of at least one eye is typically applied when the at least one eye of the user has not been corrected.

In particular, from a determination of the refractive error occurring for the user, it is possible to ascertain a spherocylindrical lens which is used as a spectacle lens to compensate the refractive errors occurring as defocusing of the at least one eye, in such a way that an image quality that is as optimal as possible can be obtained for the user. Various modes of expressions are suitable for describing the spherocylindrical lens. The standard defines in section 11.2 what is known as a “spherical power,” which is defined as a value for a vertex power of a spectacle lens with spherical power or for the respective vertex power in one of two principal meridians of the spectacle lens with astigmatic power. According to the standard, 9.7.1 and 9.7.2, the “vertex power” is defined as the reciprocal of a paraxial back vertex focal length, in each case measured in meters. The spherocylindrical spectacle lens with astigmatic power in accordance with the standard, section 12, combines a paraxial, parallel beam of light in two separate focal lines perpendicular to one another and therefore has a vertex power only in the two principal meridians. The “astigmatic power” is here defined by cylinder power and axis position. In this case, the “cylinder strength” in accordance with the standard, 12.5, represents the absolute value of an “astigmatic difference,” which indicates the difference between the vertex powers in the two principal meridians. In accordance with the standard, 12.6, the “axis position” denotes a direction of the principal meridian whose vertex power is used as a reference value. Finally, according to the standard, 12.8, the “strength” of the spectacle lens with astigmatic power is specified by means of three values, comprising the vertex powers of each of the two principal meridians and the cylinder strength.

According to L. N. Thibos, W. Wheeler and D. Homer (1997), Power Vectors: An Application of Fourier Analysis to the Description and Statistical Analysis of Refractive Error, Optometry and Vision Science 74 (6), pages 367-375, in order to describe an arbitrary spherocylindrical lens and/or the refractive error, it is suitable in each case to specify a visual acuity vector which can be described by exactly one point in a three-dimensional dioptric space, wherein the three-dimensional dioptric space can be spanned by coordinates which correspond to the visual acuity and the cylindrical strength or are correlated therewith.

According to step a) of the present method there is a representation of at least one symbol on a visual display unit, wherein at least one parameter of the at least one symbol represented on the visual display unit is varied. Here, the term “visual display unit” denotes any electronically controllable display with a two-dimensional extent, with the respectively desired symbol being representable with largely freely selectable parameters at any location within the extent. In this case, the visual display unit can typically be selected from a monitor, a screen or a display. In this case, the visual display unit can typically be contained in a mobile communications device. In this case, the term “mobile communications device” encompasses in particular a cellular phone (cellphone), a smartphone or a tablet. However, other types of mobile communications devices are conceivable. In this way, the present method for determining a refractive error of the at least one eye can be carried out at any desired location. However, other types of visual display units are likewise possible.

The term “symbol” relates firstly to at least one optotype, in particular letters, numbers or signs, and secondly to at least one pattern. While the “optotype” is an individual fixed symbol in each case, which is only able to be varied to a restricted extent in its proportions for recognition by the user, the term “pattern” denotes any graphical structure which—in particular in contrast to noise which remains without identifiable structure—has at least one spatial period, within which the structure of the pattern is represented repeatedly. Therefore, the term “periodic pattern” is also used instead of the term “pattern” in order to clearly express this property of the pattern. However, these two terms should have the same connotation below.

On account of the electronic control at least one parameter of the at least one symbol represented on the visual display unit can be varied easily and over a broad scope. The “parameter” is a property of the at least one symbol, depending on the selected symbol, in particular an extent, an intensity or a color (including black and white). In this case, the symbol, in particular the pattern, can have at least two different colors, in particular in order to be able to consider a chromatic aberration. In the case of the at least one pattern a structure can be represented repeatedly, wherein similar points or regions can form over the structure of the at least one pattern as a result of repetition. Typical configurations of similar points or regions can typically be present as periodic maxima or minima of the pattern. While the at least one selected parameter of at least one conventional optotype, in particular a letter, a number or a symbol, can therefore be an extent of the symbol, in particular a height or width, the at least one parameter in the case of the at least one periodic pattern typically relates to at least one parameter of a periodic function, in particular at least one repetition frequency. In this case, the “periodic function” denotes an instruction for a configuration of a temporally repeated, or typically spatially repeated, variation of the at least one parameter. The periodic function can typically be selected from a sine function, a cosine function, or a superposition thereof. However, other periodic functions are conceivable.

Furthermore, the at least one symbol can typically be represented on the visual display unit scaled for distance, i.e., at least one parameter of the at least one symbol can be chosen on the basis of the distance from which the user observes the at least one pattern. By way of example, in the case of a greater distance of the user from the visual display unit the at least one parameter can be chosen to be correspondingly larger. Conversely, in the case of a shorter distance of the user from the visual display unit the at least one parameter can be chosen to be correspondingly smaller.

According to the disclosure, the at least one parameter of the at least one symbol represented on the visual display unit comprises at least one spatial frequency of the at least one periodic pattern. In this case, the term “spatial frequency” denotes a reciprocal of a spatial distance between two adjacently arranged similar points, in particular a maximum or a minimum, in a spatially periodic change in the at least one pattern, which can be specified in units of 1/m or, alternatively, as a dimensionless number with the units of “units per degree” or “cycles per degree.” In this case, the at least one spatial frequency represented on the visual display unit can typically be chosen in accordance with the distance of the visual display unit from at least one eye of the user. To this end, the at least one spatial frequency represented on the visual display unit can be chosen to be higher in the case of a greater distance of the user from the visual display unit and can be chosen to be lower in the case of the smaller distance of the user from the visual display unit. In this case the intensity or the color of the at least one pattern can typically follow the curve of the periodic function, in particular the sine function, along one direction of extent of the visual display unit. Other ways of determining the spatial frequency from the at least one pattern are also conceivable however, for example from the spacing of points of equal intensity.

In a particularly typical configuration of the present disclosure, the at least one periodic pattern can be designed as a two-dimensional superposition of a periodic function, in particular the sine function, which extends in a first direction along the extent of the visual display unit and a constant function which extends in a second direction along the extent of the visual display unit, which second direction can typically be arranged to be perpendicular to the first direction. In this case the term “perpendicular” denotes an angle of 90°±30°, typically 90°±15°, particularly typically 90°±5°, in particular 90°±1°. However, other angles between the first direction and the second direction are likewise possible. In this way, the at least one pattern can be present in the form of stripes arranged next to one another in periodic fashion, which can also be referred to as a “sinusoidal grating.” However, other types of patterns are possible.

In a further typical configuration, the at least one pattern can be presented on the extent of the visual display unit in such a way that the first direction or the second direction adopts a fixed angle in relation to an orientation of the visual display unit, the respective fixed angle typically being 0° or a multiple of 90°. In this case, the term “orientation” denotes a direction which is parallel to an edge of the visual display unit which usually adopts the shape of a rectangle. In this way the at least one pattern can be adapted to the extent of the visual display unit present. However, other ways of representing the at least one pattern on the visual display unit are conceivable, for example at a fixed angle in relation to the orientation of the visual display unit of 45° or an odd multiple thereof.

In a further typical configuration, a further function, typically by at least one increasing function or decreasing function, can be superposed on the selected periodic function or the at least one periodic pattern. In this way, the amplitude of the selected periodic function can increase or decrease along the direction of the extent of the visual display unit. However, as an alternative or in addition thereto, it is particularly typical for the at least one spatial frequency of the at least one periodic pattern to increase or decrease in the direction of the extent of the visual display unit. In this way, the representation of the at least one periodic pattern on the visual display unit can already have a number of spatial frequencies, which are typically arranged in increasing order or in decreasing order in the direction of the extent of the visual display unit.

As explained in more detail below, it is alternatively or additionally possible for the periodic pattern, to be able to be represented initially in a first direction and subsequently in a second direction, which is arranged perpendicular to the first direction. In this way, the vertex power values for each of the two principal meridians, which are perpendicular to one another, can be ascertained successively for the spherocylindrical spectacle lens with astigmatic power.

According to step b) a reaction of the user is captured depending on the at least one symbol represented on the visual display unit, typically during the representation of the at least one symbol on the visual display unit as per step a). The term “reaction” denotes a response of the user to a stimulus of the at least one eye as a consequence of representing the at least one symbol on the visual display unit. The term “capture” in this case denotes recording a measurement signal which the user generates as a consequence of their one reaction. In particular, the capture of the reaction of the user during step b) can be implemented in a monocular fashion, i.e., the reaction of the user is captured individually, typically in succession, for each of the two eyes of the user. To this end, the user can typically cover the respective other eye, which is not being used. Changing the respective eye for observing the at least one symbol on the visual display unit can be prompted in this case by way of appropriate menu navigation by means of the mobile communications device, for example.

An input unit can be provided in a particularly typical configuration, the input unit being configured to capture the reaction of the user depending on the at least one symbol represented on the visual display unit. The input unit can be a keyboard, in particular a keyboard with keys which the user can operate, typically press. As an alternative or in addition thereto, this can typically be a virtual keyboard represented on a touch-sensitive visual display unit (touchscreen) of the mobile communications device, the user likewise being able to operate, typically press, the virtual keyboard. By operating the input unit the user can consequently generate a measurement signal by means of the input unit, the measurement signal being able to be transmitted to an evaluation unit as described in more detail below.

According to step c), a point of time is established typically while step b) is carried out, which point in time is defined by virtue of a recognizability of the at least one symbol represented on the visual display unit by the user being evident at the established time from the reaction of the user. The term “recognizability” in this case comprises the user just still or only just being able to recognize the at least one symbol represented on the visual display unit. If the at least one spatial frequency in the at least one periodic pattern increasingly decreases this allows the point in time to be established at which the user can only just recognize the at least one symbol represented on the visual display unit. Conversely, if the at least one spatial frequency in the at least one periodic pattern increasingly increases this allows the point in time to be established at which the user can just still recognize the at least one symbol represented on the visual display unit. In order to bring the user to carry out the reaction in the manner desired in each case, one part of the visual display unit or, alternatively or additionally, an acoustic output unit can be used to inform the user accordingly or to urge the desired reaction. In this case, the evaluation unit can establish the desired point in time at which it is evident from the reaction of the user that a recognizability of the at least one spatial frequency represented on the visual display unit by the user is given from the measurement signal which was generated during step b) by operating the input unit and which was transmitted to the evaluation unit.

According to step d), a value for the refractive error of the at least one eye of the user is determined, typically in the evaluation unit, from the value of the at least one parameter used at the established time to set the selected at least one parameter of the at least one symbol on the visual display unit, this typically being implemented following the establishment of the point in time in accordance with step c). According to the present disclosure, the value for the refractive error is determined from the at least one spatial frequency of the at least one pattern defined at the point in time at which the user has specified as per step c) that they can just still or only just identify the patterns on the visual display unit. Advantageously, the user can carry out the method proposed herein themselves in subjective fashion. In so doing, they need to rely neither on an apparatus installed at one point nor on an operator, in particular an optician.

To determine the value for the refractive error from the spatial frequency of the at least one pattern defined at the established point in time it is possible to ascertain a so-called “apparent resolution” of the user. This apparent resolution relates to a physical phenomenon which describes a contrast transfer at a resolution limit of a defocused optical system. As per M. Young, Optik, Laser, Wellenleiter, 1997, the apparent resolution of an optical system is defined in relation to the phase of an optical transfer function (OTF), which is generally non-zero. A non-zero phase indicates a spatial shift of the at least one pattern in relation to the position of the at least one pattern as predicted by geometric optics. If the phase of the OFT assumes a value of π, the image of a sinusoidal grating is shifted by half a period in relation to the geometric-optical image. This phenomenon occurs in particular when details are below the resolution limit of a defocused optical system. According to the disclosure, the physical phenomenon of apparent resolution is now used to determine the resolution limit of the at least one eye of the user in which this phenomenon occurs, and from this to calculate the defocusing that corresponds to the desired correction of the refractive error of the at least one eye of the user.

In general, the apparent resolution can be described by a Bessel function. However, according to F. Schaeffel and A. de Queiroz, Alternative Mechanisms of Enhanced Underwater Vision in the Garter Snakes Tamnophis melanogaster and T couchii, Copeia 1990 (1), pp. 50-58, the following approximation as per equation (1) can be specified for the spatial frequency which corresponds to the apparent resolution

$$Spatial frequency = \frac{21.3}{Defocus [ D ] \cdot pupil diameter [ m ]} \qquad ( 1 )$$

where the spatial frequency is specified in this case as a dimensionless number “units per degree” or “cycles per degree.” Rewriting allows the defocusing in diopters D to be ascertained herefrom as per equation (2)

$$Defocus [ D ] = \frac{21.3}{spatial frequency \cdot pupil diameter [ m ]} \qquad ( 2 )$$

which corresponds in a first approximation to the spherical equivalent of the correction.

Since as per equations (1) and (2) both the apparent resolution and the defocusing are dependent on a diameter of the pupil (pupil diameter) in at least one eye of the user, the pupil diameter is used when determining the refractive error of the at least one eye of the user. To this end, the pupil diameter can be estimated to be a value ranging from 2 to 3 mm, with this value corresponding to an average diameter of the pupil in daylight. Here, the “pupil” denotes an entry opening that is present in each eye, through which radiation in the form of light can enter into the interior of the eye. In the opposite direction, the pupil can be regarded as an exit opening, through which a viewing direction of the user from the eye to the surroundings can be defined.

Typically, however, the pupil diameter can be captured by measurement. To this end, an image of an eye area of the user can be recorded before or after, but typically during, one of the steps a) to c), in particular during step a). To this end, use can typically be made of a camera, wherein the camera can typically be included in the mobile communications device. This can be at least one rear camera or typically at least one front camera of the mobile communications device. In this way it is typically possible to record the desired image of the eye area of the user by means of the camera at any desired location. Geometric data of the pupil, in particular relative position and diameter of the pupil, can be ascertained from the recorded image, in particular by applying image processing and typically in the evaluation unit. However, other ways of determining the pupil diameter are likewise possible.

In a further configuration of the present disclosure it is consequently possible to ascertain a “pupil distance” in the case of a known distance between the at least one camera and the at least one eye of the user, wherein the pupil distance can subsequently be corrected to different distances. This distance can typically be determined by way of a distance measurement, in particular by means of a distance measurement with which the mobile communications device has already been equipped. As an alternative or in addition thereto, this distance can be determined by triangulation by way of a known number of pixels of the camera when a known object or image content is detected. Moreover, one or more wearing parameters of the user, typically the corneal vertex distance of the at least one eye of the user or the interpupillary distance between the two eyes of the user, can be determined using machine learning, typically in the evaluation unit, by evaluating photos or videos recorded by means of sensors of the mobile communications device.

As already mentioned, the spherical equivalent of the correction can be determined to a first approximation by ascertaining the apparent resolution. If the apparent resolution is determined along at least two meridians, typically by a representation of the at least one periodic pattern initially in a first direction and subsequently in a second direction arranged perpendicular to the first direction, as described in more detail above or below, this can lead to the determination of the spherocylindrical correction. For further details in this respect reference is made to WO 2018/077690 A1.

In a further configuration of the present disclosure, the at least one pattern represented on the visual display unit can be a monochromatic pattern. In this configuration, a chromatic aberration can additionally be taken into account when measuring the apparent resolution. Longitudinal chromatic aberration, in particular, leads to monochromatic light at a wavelength of approximately 450 nm being focused in front of the retina in the case of an emmetropic eye or eye rendered emmetropic by correction while monochromatic light at a wavelength greater than 600 nm is focused behind the retina. As a result of this naturally occurring defocusing, it may be advantageous for the user to set their apparent resolution, typically for both wavelengths, successively on the visual display unit. Alternatively, the user can set a mean apparent resolution while simultaneously representing the two monochromatic wavelengths.

Since from a physical point of view the defocusing leads to the same value of apparent resolution independently of its sign, a distinction can be made in a further method step as to whether the user suffers from myopia or hyperopia. In a first configuration, this can be implemented by simple questioning procedure, for example by virtue of the user being prompted to respond to a question such as “is your distance vision poor?,” typically being able to be posed by means of the mobile communications device, by entering one of the two response options of “yes” and “no.” As an alternative or in addition thereto an optical stimulus can be provided on the mobile communications device in a further configuration and the user can be prompted to provide an input. If the user cannot see the stimulus at the short distance, the assumption can be made that they are myopic. In this case, one of the configurations proposed here is particularly advantageous.

In a further configuration it is possible to take into account that the user wears a pair of spectacles with spectacle lenses and the eye of the user has a high value for the apparent resolution. In this case, further feedback from the user can be queried in respect of whether their spectacle lenses are still in order, by virtue of a further questioning procedure.

Alternatively, the user can be urged to report that they require new spectacle lenses in a further configuration where the apparent resolution has been reduced.

Overall, a so-called “psychophysical algorithm” is used in the scope of the present disclosure to determine the apparent resolution. Here the psychophysical algorithm denotes a procedure which is based on regular interactions between a subjective, mental experience of the user and quantitatively measurable, objective physical stimuli as the trigger for the experience of the user. In this case, the experience of the user consists in the specific experience of just no longer being able to identify a selected pattern, or being able to identify it for the first time. In this case, the pattern was represented by the use of an objective parameter on a visual display unit observed by the user. If the user reacts to this experience according to the prompt provided for them, by virtue of providing an appropriate input on an apparatus, it is possible to determine a quantitatively measurable variable from the input if the objective parameter in relation to the pattern is known, it being possible to determine the apparent resolution of an eye of a user from the quantitatively measurable variable in the case of the present disclosure and, from this, it being possible to determine the refractive error of the eye of the user related thereto as an objective physical variable.

In a further configuration, the individual steps of the method, listed above, for determining a refractive error of at least one eye of a user are carried out with the aid of at least one mobile communications device. Typically, at least one mobile communications device should be understood to mean an apparatus which comprises at least one programmable processor and at least one camera and at least one acceleration sensor, and which is typically designed to be carried, i.e., configured in respect of dimensions and weight so that a person is capable of carrying it along. Further components can be present in the at least one mobile communications device, for example at least one visual display unit, at least one light source for, e.g., visible light from a wavelength range of 380 nm to 780 nm and/or infrared light from a wavelength range of 780 nm to 1 mm and/or at least one light receiver with a sensitivity to, e.g., visible light from a wavelength range from 380 nm to 780 nm and/or infrared light from a wavelength range from >780 nm to 1 mm. Typical examples of such mobile communications devices, as already mentioned above, are smartphones or tablet PCs, which may comprise at least one visual display unit, for example a sensor visual display unit (touchscreen), at least one camera, at least one accelerometer, at least one light source, at least one light receiver and further components such as wireless interfaces for mobile radio and WLAN (wireless LAN). The representation of at least one symbol as per step a) of the method according to the disclosure can be implemented for example by means of the at least one visual display unit of the at least one mobile communications device.

Capturing a reaction of the user as per step b) of the method according to the disclosure can be implemented for example by means of the at least one camera or by means of the at least one light source and by means of the at least one camera, in each case of the at least one mobile communications device. Establishing a time at which a recognizability of the at least one symbol represented on the visual display unit is evident from the reaction of the user as per step c) of the method according to the disclosure can be implemented for example by means of the at least one camera or by means of the at least one light source and by means of the at least one camera, in each case of the at least one mobile terminal. Furthermore, the at least one camera of the mobile communications device can comprise at least one autofocus system. The at least one camera can have a zoom objective with a variable viewing angle or at least two objectives with different viewing angles. If the at least one camera has at least one distance sensor it is possible to determine the distance between the visual display unit of the mobile communications device and the eye of the user, for example by means of the signal from the distance sensor. If the camera has at least two objectives which may have an identical viewing angle or different viewing angles and which are spatially separated from one another in the lateral direction, it is possible to determine the distance between the camera of the mobile communications device and the eye of the user by means of a triangulation method, for example. In the latter case the viewing angle of the at least two objectives is typically identical.

In a further aspect, the present disclosure relates to a computer program for determining a refractive error of at least one eye of a user, wherein the computer program is set up to determine the refractive error of the at least one eye of the user in accordance with the method, described herein, for determining a refractive error of at least one eye of a user.

In a further aspect, the present disclosure relates to a method for producing a spectacle lens, wherein the spectacle lens is produced by processing a lens blank (standard, section 8.4.1) or a spectacle lens semifinished product (standard, section 8.4.2), wherein the lens blank or the spectacle lens semifinished product is processed on the basis of refraction data and optionally centration data, wherein the refraction data and optionally centration data comprise instructions for compensating for the refractive error of at least one eye of the user, wherein a determination of the refractive error of the at least one eye of the user is implemented in accordance with the method, described herein, for determining a refractive error of at least one eye of a user. The refraction data typically comprise the correction of the refractive error of the at least one eye of the user with respect to the spherical correction and the astigmatic correction with axis position, in each case for distance vision and/or for near vision. The centration data typically comprise at least the face form angle, the angle between the frame plane and the right or left lens plane, pursuant to the standard, section 17.3, and/or the coordinates of the centration point, i.e., the absolute value of the distance of the centration point from the nasal vertical side or from the lower horizontal side of the boxed system, measured in the lens plane, pursuant to the standard, section 17.4, and/or the corneal vertex distance, i.e., the distance between the back surface of the spectacle lens and the apex of the cornea measured in the viewing direction perpendicular to the frame plane, pursuant to the standard, section 5.27, and/or the “as-worn” pantoscopic angle or pantoscopic angle, i.e., the angle in the vertical plane between the normal with respect to the front surface of a spectacle lens at the center thereof according to the boxed system and the fixation line of the eye in the primary position, which is usually assumed as horizontal, pursuant to the standard, section 5.18, and/or optionally the far visual point, i.e., the assumed position of the visual point on a spectacle lens for distance vision under given conditions, pursuant to the standard, section 5.16, and/or optionally the near visual point, i.e., the assumed position of the visual point on a spectacle lens for near vision under given conditions, pursuant to the standard, section 5.17.

Moreover, the centration data also comprise further data which relate to a selected spectacle frame. By way of example, the pupil distance relates individually to the user while a visual point is defined by an interaction with the spectacle frame.

In a further aspect, the present disclosure relates to an apparatus for determining the refractive error of at least one eye of the user. According to the disclosure, the apparatus comprises

a visual display unit which is configured to represent at least one symbol and a change of at least one parameter of the at least one symbol;

an input unit which is configured to capture a reaction of the user depending on the at least one symbol represented on the visual display unit; and

an evaluation unit which is configured to establish a point in time at which a recognizability of the at least one symbol represented on the visual display unit by the user is evident from the reaction of the user, and to determine a value for the refractive error of the at least one eye of the user from the at least one parameter defined at the point in time,

wherein the visual display unit is configured to represent at least one periodic pattern as the at least one symbol, wherein the at least one parameter of the symbol represented on the visual display unit comprises at least one spatial frequency of the at least one periodic pattern, and wherein the evaluation unit is configured to determine the value for the refractive error of the at least one eye of the user from the at least one spatial frequency, defined at the point in time, of the at least one periodic pattern.

In a particularly typical configuration, the apparatus can furthermore comprise at least one camera, wherein the at least one camera is configured to take a recording of an image of the at least one eye of the user. In this configuration, the evaluation unit can furthermore be configured to ascertain the pupil diameter of the at least one eye of the user by applying image processing to this image and by determining a pupil distance between the at least one camera and the at least one eye of the user.

For definitions and optional configurations of the computer program and of the apparatus for determining a refractive error of at least one eye of a user and also of the method for producing a spectacle lens, reference is made to the description above or below of the method for determining a refractive error of at least one eye of a user.

The apparatus according to the disclosure and the present methods have numerous advantages over conventional apparatuses and methods. Hence, a subjective ascertainment of the correction of a refractive error of at least one eye of a user can be implemented without specialist devices and in particular can also be used by non-specialist. Furthermore, the physical phenomenon of apparent resolution is advantageously used here to ascertain the correction, allowing the defocusing of the at least one eye of the user to be determined in a simple manner.

In summary, in the context of the present disclosure, the following clauses are particularly typical:

Clause 1. A method for determining a refractive error of at least one eye of a user, wherein the method comprises the following steps:
a) representing at least one symbol on a visual display unit, wherein at least one parameter of the at least one symbol represented on the visual display unit is varied;
b) capturing a reaction of the user depending on the at least one symbol represented on the visual display unit;
c) establishing a point in time at which a recognizability of the at least one symbol represented on the visual display unit by the user is evident from the reaction of the user; and
d) determining a value for the refractive error of the at least one eye of the user from the at least one parameter defined at the point in time,

wherein the at least one symbol represented on the visual display unit is at least one periodic pattern, wherein the at least one parameter of the at least one pattern represented on the visual display unit comprises at least one spatial frequency, and wherein the value for the refractive error is determined from the at least one spatial frequency of the at least one pattern defined at the point in time.

Clause 2. The method according to the preceding clause, wherein the at least one spatial frequency of the at least one pattern is represented on the visual display unit scaled for distance.

Clause 3. The method according to either of the two preceding clauses, wherein the at least one spatial frequency of the at least one pattern represented on the visual display unit is increased or decreased.

Clause 4. The method according to the preceding clause, wherein the at least one spatial frequency of the at least one pattern represented on the visual display unit is varied over time or space.

Clause 5. The method according to any one of the preceding clauses, wherein the at least one pattern is formed by a superposition of at least one periodic function and at least one constant function.

Clause 6. The method according to the preceding clause, wherein the at least one periodic function is selected from a sine function, a cosine function or a superposition thereof.

Clause 7. The method according to any one of the preceding clauses, wherein at least one increasing function or at least one decreasing function is additionally superposed on the at least one periodic function so that the at least one spatial frequency of the at least one pattern increases or decreases in one direction.

Clause 8. The method according to the preceding clause, wherein the direction assumes an angle in relation to an orientation of the visual display unit, wherein the angle is 0° or a multiple of 90°.

Clause 9. The method according to any one of the preceding clauses, wherein the at least one pattern is initially represented in the first direction and subsequently represented in a second direction which has been varied in relation to the first direction.

Clause 10. The method according to the preceding clause, wherein the respective spatial frequency of at least one pattern in the first direction and in the second direction is used to determine a spherocylindrical correction.

Clause 11. The method according to any one of the preceding clauses, wherein the value (220) of the refractive error of the at least one eye (112) of the user (114) corresponds to a defocusing of the at least one eye (112) of the user (114), wherein the defocusing is determined as Defokus [D] in diopter D as per equation (2),

$$Defocus [ D ] = \frac{21.3}{spatial frequency \cdot pupil diameter [ m ]} \qquad ( 2 )$$

wherein the at least one spatial frequency which the user (114) can only just or just still recognize is specified is a dimensionless number and wherein the at least one eye (112) of the user (114) has a pupil diameter (156) in m.

Clause 12. The method according to any one of the preceding clauses, wherein the pupil diameter of the at least one eye of the user is estimated to be a value ranging from 2 mm to 3 mm.

Clause 13. The method according to either of the two preceding clauses, wherein the pupil diameter of the at least one eye of the user is captured by measurement.

Clause 14. The method according to any one of the preceding clauses, wherein the pupil diameter of the at least one eye of the user is ascertained by recording an image of the at least one eye of the user by means of a camera, by applying image processing to the image and by determining a pupil distance between the camera and the at least one eye of the user.

Clause 15. The method according to any one of the preceding clauses, wherein the capture of the reaction of the user during step b) is implemented in a monocular fashion, the reaction of the user being captured individually, typically in succession, for each of the two eyes of the user, wherein the user typically covers the respective other eye, which is not being used.

Clause 16. A computer program for determining a refractive error of at least one eye of a user, wherein the computer program is configured to carry out the method steps according to any one of the preceding clauses.

Clause 17. A method for producing a spectacle lens, wherein the spectacle lens is produced by processing a lens blank or a spectacle lens semifinished product, wherein the lens blank or the spectacle lens semifinished product is processed on the basis of refraction data and optionally centration data, wherein the refraction data and optionally centration data comprise instructions for compensating the refractive error of the at least one eye of the user, wherein a determination of the refractive error of the at least one eye of the user is implemented in accordance with the method steps according to any one of the preceding clauses relating to the method for determining a refractive error of at least one eye of a user.

Clause 18. An apparatus for determining the refractive error of at least one eye of the user, wherein the apparatus comprises:

a visual display unit which is configured to represent at least one symbol and a change of at least one parameter of the at least one symbol;

an input unit which is configured to capture a reaction of the user depending on the at least one symbol represented on the visual display unit; and

an evaluation unit which is configured to establish a point in time at which a recognizability of the at least one symbol represented on the visual display unit by the user is evident from the reaction of the user, and to determine a value for the refractive error of the at least one eye of the user from the at least one parameter defined at the point in time,

wherein the visual display unit is configured to represent at least one periodic pattern as the at least one symbol, wherein the at least one parameter of the at least one symbol represented on the visual display unit comprises at least one spatial frequency of the at least one periodic pattern, and wherein the evaluation unit is configured to determine the value for the refractive error of the at least one eye of the user from the at least one spatial frequency, defined at the point in time, of the at least one periodic pattern.

Clause 19. The apparatus according to the preceding clause, wherein the apparatus furthermore comprises at least one camera, wherein the at least one camera is configured to take a recording of an image of the at least one eye of the user.

Clause 20. The apparatus according to the preceding clause, wherein the evaluation unit is furthermore configured to ascertain the pupil diameter of the at least one eye of the user by applying image processing to the image of the at least one eye of the user and by determining a pupil distance between the camera and the at least one eye of the user.

Clause 21. The apparatus according to any one of the three preceding clauses, wherein the apparatus is configured as a mobile communications device, wherein the mobile communications device comprises the visual display unit, the input unit, the evaluation unit and optionally the at least one camera.

Clause 22. The apparatus according to the preceding clause, wherein the mobile communications device is configured as a smartphone.

In a further aspect, the above-described method and/or the above-described apparatus and/or the above-described computer program can be used together with at least one further method and/or at least one further apparatus and/or a further computer program. The at least one further method can be for example a method for determining a refractive error of a user's eye, typically a method in accordance with EP 3730037 A1, wherein the method comprises the following steps:
a) representing a symbol on a visual display unit, wherein a parameter of the symbol represented on the visual display unit is varied;
b) capturing an eye movement metric of the user's eye depending on the symbol represented on the visual display unit; and
c) establishing a point in time at which a recognition threshold of the user for the symbol represented on the screen is evident from the eye movement metric of the user's eye; and
d) determining a value for the refractive error of the user's eye from the parameter defined at the point in time.

As an alternative or in addition to the above-described method, the at least one further method can also be for example a method for determining at least one optical parameter of a spectacle lens, typically a method as per EP 3730998 A1, with this method comprising the following steps:
a) recording an image using a spectacle lens; and
b) ascertaining at least one optical parameter of the spectacle lens by means of image processing of the image, wherein the image comprises an eye area including the eyes and/or a facial area adjoining the eyes, of a user of the spectacle lens.

As an alternative or in addition to the methods described above, the at least one further method can for example also be a method for measuring the refractive power distribution of a left and/or a right spectacle lens in a spectacle frame, typically a method in accordance with EP 3730919 A1, in which, in a first step, at least one image capture device is used to capture at least one first imaging of a scene from at least one first recording position, wherein the at least one first imaging has at least two structure points and contains a left and/or a right spectacle lens in a spectacle frame with a section of the spectacle frame that defines a coordinate system of the spectacle frame, wherein the at least one imaging beam path for each of these at least two structure points in each case at least once passes and at least once does not pass through the first and/or the second spectacle lens of the spectacle frame. Each imaging beam path comprises the position of the structure point and also the chief ray incident in the at least one image capture device. A further step, which can temporally precede or succeed the first step, involves capturing at least one further imaging of the scene without the first and/or the second spectacle lens of the spectacle frame or without the spectacle frame containing the first and/or the second spectacle lens with the same at least two structure points of the first imaging of a scene by means of at least one image capture device from the first recording position or from at least one further recording position different than the first recording position. The at least one image capture device in the further step can be identical or different to the at least one image capture device from the first step. Typically, the at least one image capture device in the further step is identical to the at least one image capture device from the first step. Thereupon, in a calculation step, the coordinates of these at least two structure points are determined by means of image evaluation in a coordinate system, referenced to the coordinate system of the spectacle frame, of the image representation of this scene from the respective at least one beam path of these at least two structure points which has not passed the left and/or right spectacle lens in each case and the at least one further image representation of the scene. After this step, the refractive power distribution is determined in a step of determining a refractive power distribution for at least one section of the left spectacle lens in the coordinate system of the spectacle frame and/or in a step of determining a refractive power distribution for at least one section of the right spectacle lens in the coordinate system of the spectacle frame, in each case from the imaging beam paths which have passed through the respective spectacle lens.

As an alternative or in addition to the methods described above, the at least one further method can for example also be a method for measuring the refractive power distribution of a left and/or a right spectacle lens in a spectacle frame, typically a method in accordance with EP 3730919 A1, in which, in a first step, at least one image capture device is used to capture at least one first imaging of a scene from at least one first recording position, wherein the at least one first imaging has at least two structure points and contains a left and/or a right spectacle lens in a spectacle frame with a section of the spectacle frame that defines a coordinate system of the spectacle frame, wherein the at least one imaging beam path for each of these at least two structure points in each case at least once passes and at least once does not pass through the first and/or the second spectacle lens of the spectacle frame. Each imaging beam path comprises the position of the structure point and also the chief ray incident in the at least one image capture device. A further step, which can temporally precede or succeed the first step or be carried out simultaneously with the first step, involves capturing at least one further imaging of the scene with the left and/or the right spectacle lens in a spectacle frame and with a section of the spectacle frame defining a coordinate system of the spectacle frame by means of at least one image capture device from at least one further recording position different than the first recording position, with at least one imaging beam path for the same at least two structure points captured in the first imaging, wherein the at least one imaging beam path in each case at least once passes and at least once does not pass through the first and/or the second spectacle lens of the spectacle frame. That is followed by a further step which involves calculating the coordinates of the at least two structure points in a coordinate system—referenced to the coordinate system of the spectacle frame—of the scene from the respective at least one beam path of the at least two structure points which has respectively not passed through the left and/or right spectacle lens, and the at least one further imaging of the scene by means of image evaluation. Afterward, the refractive power distribution is calculated for at least one section of the left spectacle lens in the coordinate system of the spectacle frame and/or the refractive power distribution is determined for at least one section of the right spectacle lens in the coordinate system of the spectacle frame, in each case from the imaging beam paths which have passed through the respective spectacle lens.

Typically, in the two methods above for measuring the refractive power distribution of a left and/or a right spectacle lens, typically in a spectacle frame, a multiplicity of structure points are captured in the respective first imaging of a scene from in each case at least one first recording position and the respectively succeeding steps are carried out on the basis of this respective multiplicity of structure points. A multiplicity of structure points is understood to mean typically at least 10, more typically at least 100, particularly typically at least 1000 and very particularly typically at least 10,000 structure points. In particular, a multiplicity of structure points is ≥100 structure points and ≤1000 structure points.

As an alternative or in addition to the methods described above, the at least one further method can for example also be a method for determining the refractive power distribution of a spectacle lens, typically a method in accordance with EP 3730918 A1, which for example makes possible a local refractive power from the size and/or shape comparison of the imaging of the front eye section for a specific viewing direction. This is done by carrying out at least one recording of the front eye section with and without a spectacle lens situated in front of the latter, and respectively comparing the recording with and without a spectacle lens with one another.

In a superordinate application, the various methods described above, i.e., the method according to the disclosure and also the at least one further method, can be combined in order, from a comparison of the results respectively obtained, for example, to obtain a higher accuracy or a plausibility check of the results obtained in the individual methods. The various methods described above can be effected successively or simultaneously in the superordinate application. If the various methods are effected successively, their order can be independent of one another and/or any desired order can be involved. If the various methods are effected successively, preference may be given to carrying out at least one of the above-described methods for determining the refractive power distribution last. A superordinate application can be for example a computer program comprising the various methods.

Further details and features of the disclosure will become apparent from the following description of exemplary embodiments. In this case, the respective features can be realized by themselves or as a plurality in combination with one another. The disclosure is not restricted to the exemplary embodiments. The exemplary embodiments are illustrated schematically in the figures. In this case, identical reference numerals in the individual figures designate identical or functionally identical elements or elements corresponding to one another with regard to their functions.

### BRIEF DESCRIPTION OF THE DRAWINGS

FIG. 1 shows a typical exemplary embodiment of an apparatus for determining a refractive error of an eye of a user; and

FIG. 2 shows a flowchart of the method according to the disclosure for determining the refractive error of the eye of the user.

### DESCRIPTION OF EXEMPLARY EMBODIMENTS

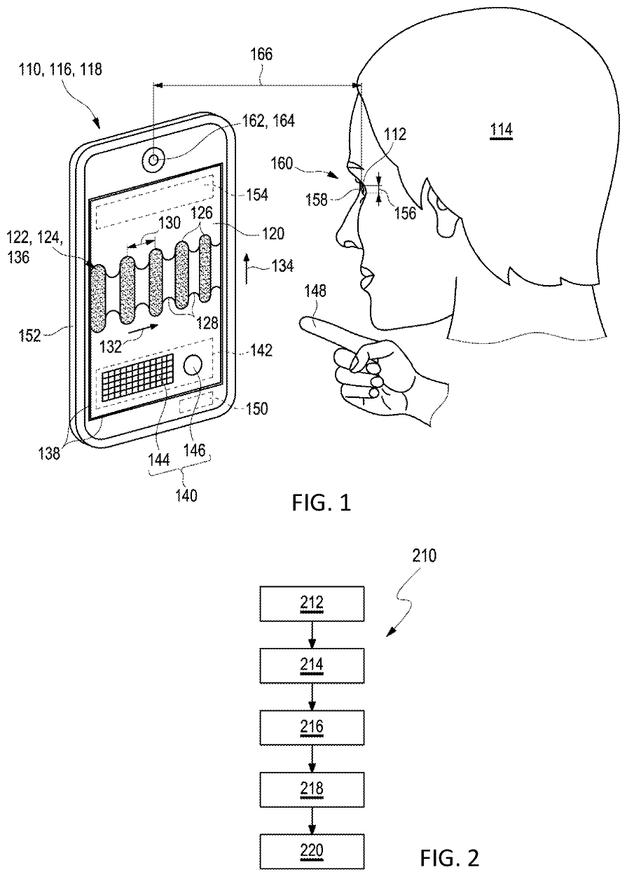

FIG. 1 schematically shows a typical exemplary embodiment of an apparatus 110 for determining a refractive error of an eye 112 of a user 114. In the illustration in accordance with FIG. 1 and in the following description—but without loss of generality—the proposed apparatus 110 is embodied as a mobile communications device 116 in the form of a smartphone 118. An embodiment of the apparatus 110 in the form of some other mobile communications device 116, in particular as a cellular phone (cellphone) or tablet, is likewise conceivable, however.

The apparatus 110 comprises a visual display unit 120, which, as is evident from FIG. 1, substantially adopts the shape of a rectangle. The visual display unit 120 is configured to represent a symbol 122. According to the disclosure the symbol 122 represents a pattern 124 which comprises a graphical structure—in particular in contrast to noise which remains without identifiable structure—which has at least one spatial period, within which the structure of the pattern 124 is represented repeatedly. The pattern is therefore also referred to as a periodic pattern.

Furthermore, the visual display unit 120 is configured to represent a change in a parameter of the symbol 122 represented on the visual display unit. On account of the electronic control of the visual display unit 120 on the smartphone 118, the selected parameter of the pattern 124 represented on the visual display unit can be varied easily and over a broad scope. In the periodic pattern 124 present here, the parameter can typically be linked to a property of a periodic function. In particular, a repetition frequency can be used in this case, with which the structure can be represented with such repetition that similar points or regions can form over the structure of the pattern 124 as a result of the repetition. In the illustration as per FIG. 1 periodic maxima 126 and minima 128 are identifiable as typical configurations of similar points or regions of the pattern 124. The periodic function used here is a sine function. However, other periodic functions are conceivable, for example a cosine function or a superposition of a cosine function on a sine function.

According to the disclosure, the parameter of the symbol represented on the visual display unit 120 comprises at least one spatial frequency of the periodic pattern 124, wherein the term spatial frequency a reciprocal of a spatial distance 130 between adjacently arranged similar points, in particular between adjacent maxima 126 or between adjacent minima 128, in a spatially periodic change of the pattern. In this case, the spatial frequency can be specified in units of 1/m or alternatively as dimensionless number in “units per degree” or “cycles per degree.” As illustrated schematically in FIG. 1, the intensity of the pattern 124 in a first direction 132 of the extent of the visual display unit 120 can follow the curve of the periodic function, in particular the sine function, in this case. Other ways of determining the spatial frequency from the pattern are conceivable however, for example from the spacing of points of equal intensity.

As FIG. 1 furthermore shows, the periodic pattern in this particularly typical embodiment comprises a two-dimensional superposition of the periodic function, in particular the sine function, which extends in the first direction 132 along the extent of the visual display unit 120 and a constant function which extends in a second direction 134 along the extent of the visual display unit 120, which is arranged perpendicular to the first direction 132 in this case. However, other angles between the first direction 132 and the second direction 134 are likewise possible. In this way, the pattern 124 on the visual display unit 120 can be present in the form of stripes 136 arranged next to one another in periodic fashion, which are also referred to as the “sinusoidal grating.” However, other types of patterns 124 are likewise possible. The user 114 consequently observes the sinusoidal grating on the visual display unit from a defined distance, the sinusoidal grating comprising the stripes 136 arranged next to one another in periodic fashion with a high contrast and a multiplicity of spatial frequencies. A value ranging from 25 cm to 1 m, particularly typically from 40 cm to 75 cm, in particular a value of 50 cm, can typically be chosen for the distance.

In the case of nearsightedness (myopia) of the user 114, the eye 112 is defocused and such a value can be set for the distance. The same is implemented in the case of young myopic users wearing spectacles. The apparent resolution is very high in the case of a pair of spectacles which corrects a refractive error present sufficiently well. However, if the spectacles are only partially correcting, the eye 112 of the user 114 is defocused and such a value can be set for the distance. In the case of a young, farsighted (hyperopic) user 114, no measurement can be implemented in this way since a high level of residual accommodation of the eye 112 of the young user 114 does not allow any evidence of defocusing. In this case, the pattern 124 can be represented at a distance of at least 4 m; in this case, the smartphone 118 can be used as an input unit.

As is furthermore recognizable from FIG. 1 the pattern 124 can be represented on the extent of the visual display unit 120 in such a way that the first direction 132 and the second direction 134 can each be arranged parallel to an orientation of the visual display unit 120 parallel to an edge 138 of the visual display unit 120, which substantially adopts the shape of a rectangle in the illustration as per FIG. 1. In this way the pattern 124 can be adapted to the extent of the visual display unit 120 present. However, other ways of representing the pattern 124 on the visual display unit 120 are also conceivable, for example at an angle of 45°±15° or an odd multiple thereof in relation to the orientation of the visual display unit 120.

As FIG. 1 furthermore illustrates, a further function can be superposed on the selected periodic function. In the illustration as per FIG. 1, a decreasing function has been superposed on the sine function in relation to the first direction 132 in such a way that the spatial frequency of the periodic pattern 124 reduces along the first direction 132. In this way the representation of the pattern 124 on the visual display unit 120 can already have a number of spatial frequencies, which are arranged in a decreasing sequence in the first direction 132 in this case.

The apparatus 110 furthermore comprises an input unit 140 which is configured to capture a reaction of the user 114 depending on the symbol 122 represented on the visual display unit 120. In particular, the reaction of the user 114 can be captured in monocular fashion during, typically successively for each of the two eyes 112 of the user 114, wherein the user 114 can in each case cover the other eye 112, which is not being used. In this case, it is typically first the right eye 112 and subsequently the left eye 112 of the user that can be used to capture their reaction. In this case, the user can be prompted to change the eye 112 for observing the symbol 122 on the visual display unit 120, typically by way of appropriate menu navigation on the smartphone 118.

To allow the user 114 to facilitate a desired response to a stimulus of the eye 112 of the user 114 as a consequence of the representation of the symbol 122 on the visual display unit 120, the smartphone 118 can have an input area 142 in the embodiment as per FIG. 1, the input area being represented on the touch-sensitive visual display unit 120 (touchscreen) of the smartphone 118. In this case, the input area 142 can adopt the form of a virtual keyboard 144. As an alternative or in addition thereto, the input area 142 can however have a different type of configuration, for example in the form of a button 146. However, as a further alternative, provision can also be made for the input unit 140 to be attached away from the visual display unit 120 of the apparatus 110. As an alternative or in addition thereto, the input unit 140 can furthermore be configured to record a speech input, by means of which the user 114 can transmit the input of their reaction to the apparatus 110.

Independently of the actual type of embodiment of the input unit 140 the user 114 can consequently operate the input unit 140, typically by manual impingement of the input unit 140, in particular by means of a finger 148 of user 114, in such a way that the input unit 140, as a consequence of the impingement of the input unit 140 by the user 114, generates a measurement signal which can be transmitted to an evaluation unit 150 of the device 110. In a typical embodiment the user 114 can now set the spatial frequency at which they can just still recognize a black-white contrast; this corresponds to a first zero of the sine function represented on the visual display unit 120. This can be implemented, in particular, by virtue of a high spatial frequency being represented at the outset and the latter then being incrementally reduced, or by virtue of a low spatial frequency being represented at the outset and the latter then being incrementally increased. In this case, the value for the spatial frequency can be specified independently of the user 114. As an alternative or in addition thereto, the user 114 can be provided with the option of themselves influencing the spatial frequency represented on the visual display unit 120, in particular by way of actuating the input unit 140. Moreover, information as to whether the user 114 observes the visual display unit 120 with or without a visual aid can also be captured, typically likewise by actuating the input unit 140.

As illustrated schematically in FIG. 1, the apparatus 110 can furthermore have a housing 152, which may comprise the evaluation unit 150. As an alternative or in addition thereto, the evaluation unit 150 may, however, also be attached outside of the housing 152, wherein a wired or wireless connection (not illustrated) may be provided between the input unit 140 and the evaluation unit 150. However, further types of the implementation are possible.

According to the disclosure, the evaluation unit 150 is configured to determine a point in time at which a recognizability of the symbol 122 represented on the visual display unit 120 by the user 114 is evident from the reaction of the user 114, which should be understood to mean that the user 114 can only just still or only just recognize the spatial frequency of the periodic pattern 124 presented on the visual display unit. To this end, the spatial frequency in the periodic pattern 124 can increase or decrease in time and/or in space, in particular in the first direction 132. At the same time, the user 114 is urged to specify by way of an operation of the input unit 140 that they can just still or only just recognize the spatial frequency of the periodic pattern 124 represented on the visual display unit. In order to receive the reaction of the user 114 in the desired manner where possible, a display part 154 of the visual display unit 120 or, as an alternative or in addition thereto, an acoustic output unit (not illustrated) can be used to inform the user 114 accordingly or to urge the desired reaction.

According to the disclosure, the evaluation unit 150 is furthermore configured to determine a value for the refractive error of the eye 112 of the user 114 from a specification of the point in time at which it is evident from the reaction of the user 114 that the user 114 can just still or only just recognize the spatial frequency of the periodic pattern 124 represented on the visual display unit. To this end, the measurement signal generated by the user 114 during step b) by operating the input unit 140 is transmitted to the evaluation unit 150 which is configured to establish the desired point in time therefrom. Furthermore, on account of the electronic control of the visual display unit 120 on the smartphone 118, the spatial frequency of the periodic pattern 124 represented on the visual display unit 120 is known and can consequently be used by the evaluation unit 150 for the desired evaluation. To this end, the evaluation unit 150 in a particularly typical embodiment can furthermore be configured to set the desired parameter of the symbol 122, in particular the spatial frequency of the periodic pattern 124, by controlling the visual display unit 120.

To determine the value for the refractive error of the eye 112 of the user 114 from the spatial frequency of the periodic pattern 124 defined at the point in time, the determination of the apparent resolution of the eye 112 of the user 114 illustrated above is resorted to according to the disclosure. In the embodiment of the present disclosure according to FIG. 1, it is possible to this end to proceed as described above. As per equation (2) specified above

Defocus [D]=21.3/spatial frequency·pupil diameter [m] (2)

the defocusing which corresponds in a first approximation to the spherical equivalent of the sought-after correction can be ascertained in diopter D.

As per equation (2), however, the defocusing is dependent on a pupil diameter 156 of a pupil 158 in the eye 112 of the user 114. An average diameter of the pupil 158 in daylight ranging from 2 to 3 mm can be used as an estimated value for the pupil diameter 156. Typically, however, the pupil diameter 156 can be captured by measurement. To this end, an image of an eye area 160 of the user 114 can be recorded, in particular while the user 114 observes the sinusoidal grating on the visual display unit 120 of the smartphone 118. As illustrated schematically in FIG. 1, a camera 162 can typically be used to this end, wherein the camera 162 can typically be a front camera 164 of the smartphone 118.

Consequently, the desired image of the eye area 160 of the user 114 can be recorded by means of the camera 162 at any desired location. Geometric data of the pupil 158, in particular a relative position and the diameter 156 of the pupil 158 in the eye 112 of the user 114, can be ascertained from the recorded image, in particular by means of image processing which can typically be carried out by the evaluation unit 150.

If the spatial frequency of the periodic pattern 124 represented on the visual display unit 120 is known and if the pupil diameter 156 is known, the defocusing of the eye 112 of the user 114 can therefore be ascertained in diopters D using equation (2), the defocusing, as specified above, corresponding in a first approximation to the spherical equivalent of the sought-after correction. In a further embodiment it is possible to additionally ascertain a distance, referred to as pupil distance 166, between the camera 162 and the eye 112 of the user 114. A distance measurement can be performed to determine the pupil distance 166, typically a distance measurement already available in the smartphone 118. As an alternative or in addition thereto, the pupil distance 166 can be determined by triangulation by way of a known number of pixels of the camera 162 when a known object or image content is detected by the camera 162.

As already mentioned, the spherical equivalent of the correction can be determined to a first approximation by ascertaining the apparent resolution. However, the apparent resolution can also be determined along at least two meridians, typically by virtue of the periodic pattern 124 as per FIG. 1 being represented on the visual display unit 120, initially along the first direction 132 and, following this, (not illustrated) along the second direction 134 which is typically arranged perpendicular to the first direction 132 on the visual display unit 120. In this way, the vertex power values for each of the two principal meridians, which are perpendicular to one another, can be ascertained successively for the spherocylindrical spectacle lens with astigmatic power. For further details in this respect reference is made to WO 2018/077690 A1.

FIG. 2 schematically shows a flowchart of a typical exemplary embodiment of a method 210 according to the disclosure for determining the refractive error of the eye 112 of the user 114.

In a representation step 212 there is to this end, as per step a), the representation of the periodic patter 124 on the visual display unit 120, wherein the one spatial frequency of the periodic pattern 124 represented on the visual display unit 120 is varied.

In a capture step 214 there is, as per step b), the capture of the reaction of the user 114 depending on the spatial frequency of the period pattern 124 represented on the visual display unit 120 in accordance with the representation step 212.

In an establishment step 216 there is, as per step c), the establishment of the point in time at which a recognizability of the symbol 122 represented on the visual display unit 120 by the user 114 is evident from the reaction of the user 114 in the capture step 214, such that the user 114 can just still or only just recognize the spatial frequency of the periodic pattern 124 represented on the visual display unit 120 as per the representation step 212.

In a determination step 218 there is, as per step d), the determination of a value 220 for the refractive error of the eye 112 of the user 114 from the spatial frequency of the period pattern 124 defined for representing the periodic pattern 124 on the visual display unit 120 in the representation step 212 at the point in time ascertained in the establishment step 216.

The foregoing description of the exemplary embodiments of the disclosure illustrates and describes the present invention. Additionally, the disclosure shows and describes only the exemplary embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

### LIST OF REFERENCE SIGNS

110 Apparatus
112 Eye
114 User
116 Mobile communications device
118 Smartphone
120 Visual display unit
122 Symbol
124 Pattern
126 Maximum
128 Minimum
130 Spatial distance
132 First direction
134 Second direction
136 Stripes
138 Edge
140 Input unit
142 Input area
144 Keyboard
146 Button
148 Finger
150 Evaluation unit
152 Housing
154 Display part
156 Pupil diameter
158 Pupil
160 Eye area
162 Camera
164 Front camera
166 Pupil distance
210 Method for determining a refractive error of an eye of a user
212 Representation step
214 Capture step
216 Establishment step
218 Determination step
220 Value of a refractive error of a user's eye

1. A method for determining a refractive error of one or both eyes of a user, the method comprising:
representing at least one periodic pattern on a visual display unit, wherein at least one parameter of the at least one periodic pattern represented on the visual display unit includes at least one spatial frequency, and wherein the spatial frequency is varied;
capturing, with an input unit, a reaction of the user depending on the at least one periodic pattern represented on the visual display unit;
establishing, with an evaluation unit, a point in time at which a recognizability of the at least one periodic pattern represented on the visual display unit by the user is evident from the reaction of the user; and
determining, with the evaluation unit, a value for the refractive error of the one or both eyes of the user from the established point in time, wherein the value for the refractive error is determined from the at least one spatial frequency of the at least one periodic pattern defined at the established point in time,
wherein the input unit is selected from a keyboard or a touch-sensitive visual display unit of a mobile communications device.

2. The method as claimed in claim 1, wherein the at least one spatial frequency of the at least one periodic pattern is increased or decreased.

3. The method as claimed in claim 1, wherein the at least one periodic pattern is formed by a superposition of at least one periodic function and at least one constant function.

4. The method as claimed in claim 3, wherein the at least one periodic function is selected from at least one sine function, at least one cosine function, or a superposition thereof.

5. The method as claimed in claim 3, wherein at least one increasing function or at least one decreasing function is additionally superposed on the at least one periodic function so that the at least one spatial frequency of the at least one periodic pattern increases or decreases in a direction.

6. The method as claimed in claim 5, wherein the direction assumes an angle in relation to an orientation of the visual display unit, and wherein the angle is 0° or a multiple of 90°.

7. The method as claimed in claim 1, wherein the at least one periodic pattern is initially represented in the first direction and subsequently represented in a second direction which has been varied in relation to the first direction.

8. The method as claimed in claim 7, wherein the respective spatial frequency of the at least one periodic pattern in the first direction and in the second direction is used to determine a spherocylindrical correction.

9. The method as claimed in claim 1, wherein the value of the refractive error of the one or both eyes of the user corresponds to a defocusing of the one or both eyes of the user, wherein the defocusing is determined as Defocus [D] in diopter D as per equation (2),
Defocus [D]=21.3/spatial frequency·pupil diameter [m] (2)
wherein the spatial frequency which the user can only just recognize or just still recognize is specified as a dimensionless number, and wherein the respective eye of the user has the pupil diameter in m.

10. The method as claimed in claim 9, wherein the pupil diameter of the one or both eyes of the user is captured by measurement, wherein the pupil diameter of the one or both eyes of the user is ascertained by recording an image of the one or both eyes of the user with a camera, by applying image processing to the image and by determining a pupil distance between the camera and the one or both eyes of the user.

11. A computer program for determining a refractive error of one or both eyes of a user, the computer program being stored on a non-transitory storage medium and being configured to cause a computer to:
represent at least one periodic pattern on a visual display unit, wherein at least one parameter of the at least one periodic pattern represented on the visual display unit includes at least one spatial frequency, and wherein the spatial frequency is varied;
capture, with an input unit, a reaction of the user depending on the at least one periodic pattern represented on the visual display unit;
establish, with an evaluation unit, a point in time at which a recognizability of the at least one periodic pattern represented on the visual display unit by the user is evident from the reaction of the user; and
determine, with the evaluation unit, a value for the refractive error of the one or both eyes of the user from the established point in time, wherein the value for the refractive error is determined from the at least one spatial frequency of the at least one periodic pattern defined at the established point in time,
wherein the input unit is selected from a keyboard or a touch-sensitive visual display unit of a mobile communications device.

12. A method for producing a spectacle lens, which is implemented by processing a lens blank or a spectacle lens semifinished product, wherein the lens blank or the spectacle lens semifinished product is processed based on refraction data and, optionally, centration data, wherein the refraction data and optionally the centration data contain instructions for compensating a refractive error of one or both eyes of the user, and wherein the production of the spectacle lens includes a determination of the refractive error of the one or both eyes of the user, the method comprising:
representing at least one periodic pattern on a visual display unit, wherein at least one parameter of the at least one periodic pattern represented on the visual display unit includes at least one spatial frequency, and wherein the spatial frequency is varied;
capturing, with an input unit, a reaction of the user depending on the at least one periodic pattern represented on the visual display unit;
establishing, with an evaluation unit, a point in time at which a recognizability of the at least one periodic pattern represented on the visual display unit by the user is evident from the reaction of the user; and
determining, with the evaluation unit, a value for the refractive error of the one or both eyes of the user from the established point in time, wherein the value for the refractive error is determined from the at least one spatial frequency of the at least one periodic pattern defined at the established point in time,
wherein the input unit is selected from a keyboard or a touch-sensitive visual display unit of a mobile communications device.

13. An apparatus for determining a refractive error of one or both eyes of a user, the apparatus comprising:
a visual display unit configured to represent at least one periodic pattern and a change in at least one parameter of the at least one periodic pattern, wherein the at least one parameter includes at least one spatial frequency of the at least one periodic pattern;
an input unit configured to capture a reaction of the user depending on the at least one periodic pattern represented on the visual display unit; and
an evaluation unit configured to establish a point in time at which a recognizability of the at least one periodic pattern represented on the visual display unit by the user is evident from the reaction of the user,
wherein the evaluation unit is further configured to determine a value for the refractive error of the one or both eyes of the user from the established point in time,
wherein the value for the refractive error of the respective eye of the user is determined from the at least one spatial frequency of the at least one pattern defined at the established point in time, and
wherein the input unit is selected from a keyboard or a touch-sensitive visual display unit of a mobile communications device.

14. The apparatus as claimed in claim 13, further comprising:
at least one camera configured to record an image of the one or both eyes of the user, wherein the evaluation unit is further configured to ascertain the pupil diameter of the one or both eyes of the user by applying image processing to the image of the one or both eyes of the user and to determine a pupil distance between the at least one camera and the one or both eyes of the user.

15. The apparatus as claimed in claim 13, wherein the input unit, by way of an operation of the input unit, is configured to generate a measurement signal which is transmitted to the evaluation unit.
